(19) United States
(12) Patent Application Publication
(10) Pub. No.: US 2025/0204774 A1
(43) Pub. Date: Jun. 26, 2025

(54) Method and Device for Wave Front Measurement in the Human Eye

(71) Applicants: Vasyl Molebny, Houston, TX (US); Joe S. Wakil, Houston, TX (US); Theresa Sievert, Spring, TX (US); Chris Pope, Splendora, TX (US)

(72) Inventors: Vasyl Molebny, Houston, TX (US); Joe S. Wakil, Houston, TX (US); Theresa Sievert, Spring, TX (US); Chris Pope, Splendora, TX (US)

(73) Assignee: Tracey Technologies Corp., Houston, TX (US)

(21) Appl. No.: 19/064,925

(22) Filed: Feb. 27, 2025

Related U.S. Application Data
(63) Continuation of application No. PCT/US2023/073370, filed on Sep. 1, 2023.
(60) Provisional application No. 63/403,686, filed on Sep. 2, 2022.

(51) Int. Cl. A61B 3/10

(52) U.S. Cl. CPC A61B 3/1015

(57) ABSTRACT

Provided herein are a method and device for a wave front measurement in a human eye with respect to its objectively determined visual axis where the visual axis is determined prior to measuring the wave front. The visual axis is defined as a direction to the voveola of the bisectrix of two orthogonal laser beam doblets hitting the foveola in the points of equal depths of the foveola slopes. The wave front is measured by probing the eye with a laser beam oriented along the visual axis at a set of points in the pupil. Backscattered radiation is detected, the coordinates of the laser spots on the retina are measured, and the wave front is reconstructed from the refraction distribution over the pupil.

### CROSS-REFERENCE TO RELATED APPLICATIONS

This application is a continuation under 35 U.S.C. § 120 and 35 U.S.C. § 365(c) of pending international application PCT/US23/73370, filed Sep. 1, 2023, which claims benefit of priority under 35 U.S.C. § 119(e) of provisional application U.S. Ser. No. 63/403,686, filed Sep. 2, 2022, now abandoned, the entireties of which are hereby incorporated by reference.

### BACKGROUND OF THE INVENTION

### Field of the Invention

The present invention relates generally to the fields of ophthalmic instruments that are used to examine the eye. More specifically, the present invention relates to ophthalmic examination instruments that measure and characterize the refractive properties of the eye with high accuracy of their objectively determined localization.

### Description of the Related Art

If the human eye were a perfect optical system, its optical axis would be defined by points of crossing the centers of its optical components—the cornea and the crystalline lens. As the eye is not a perfect optical system, the term “optical axis of the eye” could mean only a rough approximation, that does not suit for accurate description of the features important for diagnosing and surgery. Pupillary axis and line of sight are other approximations.

When videokeratography was the only diagnostic means, centering procedures were concentrated on the corneal vertex using the pupillary axis as determined by the center of Purkinje reflexes. With the advent of wavefront sensing that involved the line of sight for measurement of aberrations of the eye, it appeared that when the value of the angle between the line of sight and the pupillary axis is larger than 2°-3°, the misalignment, if ignored, can lead to incorrect estimates of corneal and internal aberrations as well as—to incorrect estimates of the corneal/internal aberration balance, and as a consequence, may result in correction errors.

The recommendations that followed in the ANSI standard Z80.28-2004, and later in the International standard ISO/FDIS 24157:2008 (E), specified the wavefront measurements to be referenced to the center of the pupil using the line of sight as the reference axis. The appealing argument was the easy designation of the center of the entrance pupil, and keeping in mind that the visual axis has no constructive referencing to anchor the measuring instrument to the eye.

The definition of the visual axis includes two straight-line fractions: the “fixation point—first nodal point” and the “second nodal point—center of the foveola”. Because of the very small distance between the nodal points, this definition is often simplified to the “fixation point—nodal point—center of the foveola”. Since the internal fraction of the visual axis is practically the extension of its external fraction, this definition can be simplified even more: “fixation point—center of the foveola”. There is no evidence that in an axially asymmetric optical system of the eye, this straight line “fixation point—center of the foveola” crosses the pupil in the center of Purkinje reflexes, or in the geometrical center of the pupil, because its shape can affect the location of the center.

Having different reference axes in corneal topography, in optical coherence tomography, in aberrometry, in refractometry, causes difficulties when trying to integrate the data from separate instruments. The major reason for this is the current misconception among many laser companies and surgeons that ablations are centered on the entrance pupil center instead of the visual axis. Reinstein et al. made an attempt in the U.S. Pat. No. 8,444,632, to define the visual axis as an axis of least aberrations measured through a virtually created pupil whose center is anchored to the coordinates of the actual pupil. This attempt was extended by Wakil et al. in U.S. Pat. No. 9,271,647 where the procedure of mathematical search of a virtual pupil was replaced by iterative ray tracing to localize the axis with the minimum of aberrations, accepted as an axis of best vision. Both of these solutions actually replace the visual axis by its approximation, still called the “visual axis”, that should have, by suggestion, the same minimal aberrations as suggested for the actual visual axis, and should result in better outcome of surgery in comparison to the one based on centering around the line of sight.

In Ukrainian Patent No. 114043, Molebny proposed to localize the visual axis in accordance with its text-book definition where the foveola is designated by its deepest point. The direction on this point is searched by the reconstruction of the foveola's pit topography as described in the publication (V. Molebny. “Method of locating the visual axis objectively”. Ophthalmic and Physiological Optics. 2017; 37, 326-332, doi: 10.1111/opo).

Determining the point of the corneal intersection by the visual axis allows one to take into account a corresponding shift of eye parameters, and to use these data to measure any other characteristics of the eye, or to make the vision correction by means of the corneal tissue ablation, or to position the inlays, or to replace the crystalline lens by an intraocular lens properly oriented.

The method of Molebny described in Ukrainian Patent No. 114043 needs certain time to reconstruct point-by-point the retinal topography in the region of the foveola. There is a recognized need to make this procedure faster.

The prior art is deficient in the lack of instruments and methods that measure and characterize the refractive properties of the eye with high accuracy. The present invention fulfills this longstanding need and desire in the art.

### SUMMARY OF THE INVENTION

The present invention provides a method and device for wave front measurement of the human eye with respect to its objectively determined visual axis. The methods of the present invention measure the refraction parameters of the eye using a ray tracing method in which the optical axis of the device is oriented along the visual axis of the eye, this latter being defined as a straight line connecting a fixation point with the center of the foveola. The center of the foveola is determined as the deepest point in the foveola's pit, the direction to which is pointed out by the bisectrix of two laser beams abutting symmetrically on the opposite pit's slopes.

The procedure of wave front measurement is provided by measurement of the refraction parameters of the eye by ray tracing with the laser beam, deflected by two-axis acousto-optical deflector after passing a telescope and a collimating lens, being directed into the eye. The light backscattered from the retina, is detected by position sensing detectors. Refraction parameters of the eye are calculated by a processing unit.

To start these measurements, the direction of the visual axis is first determined. By a command from a processing unit, the laser beam is split (“doubled”) consecutively in X and in Y directions, providing thus a possibility to define the direction toward the foveola's pit center by their bisectrix. The splitting of the beam is provided with the same acousto-optical deflectors by applying two different frequencies to each of them having their difference corresponding to the angle of beam splitting that is of the order of 10 mrad.

The search of the bisectrix position designating the direction of the visual axis is provided by measurement of a phase difference between the pairs of the split beams, separately in X and in Y directions. This phase difference is measured on the difference frequency applied to the acousto-optical deflectors. The reference signal for phase discrimination is taken from the generators, driving the deflectors. The phase of the reference signal is compared to the phase of the low-frequency component filtered at the exit of the coherent detector receiving the signals corresponding to the split beams after their backscattering from the retina. The search of the bisectrix position is controlled by the processing unit.

This search starts after the conjugation of the retina with the planes of detectors has been made using an electrically controlled conjugating telescope installed in front of the eye. Measurement of refraction parameters is provided with the optical axis of the device oriented along the visual axis of the eye. The details of the schematic and the details of functioning, other and further aspects, features, and advantages of the present invention will be apparent from the following description of the presently preferred embodiments of the invention given for the purpose of disclosure.

### BRIEF DESCRIPTION OF THE DRAWINGS

So that the above-recited features of the invention are to be understood in detail, more particular descriptions of the invention briefly summarized above are illustrated in the appended drawings. These drawings form a part of the specification. However, that the appended drawings illustrate preferred embodiments of the invention, they are not to be considered limiting in their scope.

FIG. 1 illustrates the definitions of the visual axis of the eye, the pupillary axis, the axis of symmetry (known also as the optical axis), the line of sight, the angle κ (kappa) between the visual axis and the pupillary axis, the angle λ (lambda) between the line of sight and the pupillary axis.

FIG. 2 shows the designation of the center of the pupil with a cross-like mark as a geometrical center of a set of first Purkinje reflexes from the exterior surface of the cornea.

FIG. 3 illustrates the visual axis of the eye crossing the center of the pit in the zone of foveola centralis.

FIG. 4 is a functional schematic of a device illustrating the wave front measurement with regards to the visual axis determined by the device.

FIGS. 5A-5E illustrate the steps of forming the probing laser beams. FIG. 5A shows two orders of beam diffraction in X and Y directions, where the first digit designates the order of diffraction in the X direction, the second digit designates the order of diffraction in the Y direction where the probing uses the first order of diffraction for both X and Y directions. FIG. 5B demonstrates the spatial filtering for selection of the beams in the first order of diffraction. FIG. 5C demonstrates positioning of single beams in the mode of wave front measurement. FIG. 5D demonstrates the beam splitting (double-beam forming) in the X direction in the mode of visual axis designation. FIG. 5E demonstrates the same as FIG. 5D for the Y direction.

FIG. 6 demonstrates the paths of a doublet of laser beams (split in X direction) from the laser 1 to the collimating lens 15 (no scaling) in the schematic of FIG. 4 where the lenses are replaced by the principal planes of their thin equivalents. Definition of the bisectrix is applied from the point of splitting along the whole path till the retina.

FIGS. 7A-7C show the paths of a doublet of laser beams after the collimating lens to the eye. FIG. 7A shows an emmetropic eye where the fluidic lens is in its initial position corresponding to the requirement of afocality of the telescope 18-20. FIG. 7B shows a myopic eye where focus of the lens 18 is shifted to a longer value. FIG. 7C shows a hyperopic eye where focus of the lens 18 is shifted to a shorter value. In both cases (FIG. 7B and FIG. 7C) ametropia of the eye is to be compensated.

FIG. 8 shows the position of the point C_{VA} of crossing the cornea by the visual axis dislocated at X_{VA}, Y_{VA} in regards to the point C_{LOS}, being the center of Purkinje images, suggested to be the point of crossing the cornea by the line of sight.

FIG. 9 shows the paths of the beam doublet (split in X direction) in the zone of destination—fovea centralis. The depth of light penetration into the tissues of retina depends on the wavelength; still, for the beams shifted by Fₓ₁ and Fₓ₂ the difference in penetration does not play any role.

FIGS. 10A-10C show how the phase difference φ measured by the phase discriminator 29 of the schematic FIG. 4 can serve as an error signal for positioning the bisectrix to coincide with the visual axis of the eye. In FIG. 10A when φₓ>0, the signal error Δx is also >0, in FIG. 10B when φₓ<0, the signal error Δx is also <0 and in FIG. 10C when φₓ=0, the bisectrix coincides with the visual axis. Similar dependences work for Y direction.

### DETAILED DESCRIPTION OF THE INVENTION

As used herein, the articles “a” and “an” when used in conjunction with the term “comprising” in the claims and/or the specification, may refer to “one”, but it is also consistent with the meaning of “one or more”, “at least one”, and “one or more than one”. Some embodiments of the invention may consist of or consist essentially of one or more elements, components, method steps, and/or methods of the invention.

As used herein, the term “or” in the claims refers to “and/or” unless explicitly indicated to refer to alternatives only or the alternatives are mutually exclusive, although the disclosure supports a definition that refers to only alternatives and “and/or”.

As used herein, the terms “comprise” and “comprising” are used in the inclusive, open sense, meaning that additional elements may be included.

As used herein, the terms “consists of” and “consisting of” are used in the exclusive, closed sense, meaning that additional elements may not be included.

As used herein, the term “includes” or “including” is used herein to mean “including, but not limited to”. The terms “includes”, “including” and “including but not limited to” are used interchangeably.

As used herein, the term “about” refers to a numeric value, including, for example, whole numbers, fractions, and percentages, whether or not explicitly indicated. The term “about” generally refers to a range of numerical values (e.g., ±5-10% of the recited value) that one of ordinary skill in the art would consider equivalent to the recited value (e.g., having the same function or result). In some instances, the term “about” may include numerical values that are rounded to the nearest significant figure.

In one embodiment of the present invention, there is provided a method for measuring a wave front in a human eye with respect to its objectively determined visual axis, comprising probing the eye with a laser beam consecutively in time in a set of points within a pupil of the eye; detecting radiation backscattered from a retina in the eye; measuring coordinates of laser spots on the retina; and reconstructing the wave front from a refraction distribution over the pupil of the eye; wherein the laser beam probing the eye is oriented along the visual axis of the eye during measuring the wave front, where the visual axis is determined prior to measuring the wave by the steps of: splitting the laser beam into a beam doublet separately in X and Y directions; probing the retina in a zone of a foveola pit with the beam doublet in both the X and Y directions of beam splitting; detecting the laser light backscattered from the zone of the foveola pit; measuring and comparing a phase shift of the beams in the beam doublet relative to each other in both of the X and Y directions of beam splitting; determining the direction of the beam doublet in a position of equal depths of opposite slopes of the foveola pit indicated by a zero value of the phase shift between the beams in the beam doublet; and designating the visual axis as a bisectrix of the beam doublet in the position of equal depths of the opposite slopes of the foveola pit for both of the X and Y directions of beam splitting.

In this embodiment, the beam splitting may comprise diffracting in a two-axis acousto-optical deflector separately in orthogonal directions in a plane of the pupil. Further in this embodiment, the beam splitting may comprise diffracting in a two-axis acousto-optical deflector separately in orthogonal directions in a plane of the pupil. In addition, the opposite slopes of the foveola pit may have a depth difference thereon defined as a phase difference between carrier frequencies of the split beams converted to a phase difference between signals at a frequency difference between the carrier frequencies of said beam doublet. Furthermore, the probing with the beam doublet may be varied directionally until a value of the phase difference becomes zero for both orthogonal directions.

In another embodiment of the present invention, there is provided a device for a wave front measurement in a human eye with respect to its objectively determined visual axis, comprising a laser configured to emit a laser beam of a wavelength suitable for ray tracing; a pair of diffraction-based deflectors composed of acousto-optical crystals and comprising a first deflector oriented to deflect the laser beam in a first direction orthogonal to the laser beam propagation and a second deflector oriented to deflect the laser beam in a second direction orthogonal to the first direction, each of the first deflector and the second deflector in the pair having an entrance aperture and an exit aperture and each of the first deflector and the second deflector in the pair positioned along a path of the laser beam such that their effective centers of deflection substantially coincide; a pair of drivers comprising a first driver operably connected to a first frequency generator and a second driver operably connected to a second frequency generator, each of the first driver and the second driver in the pair configured to acousto-optically drive the first deflector and the second deflector, respectively; a telescope and a collimating lens placed sequentially in optical alignment with the centers of deflection of the first deflector and said second deflector in the pair of deflectors; a first beam splitter placed in the path of the laser beam to the human eye and of the reflected radiation from the human eye; a position sensing detector placed in the path of the reflected radiation from the human eye; and a processing unit in operable communication with at least the laser, the first frequency generator, the second frequency generator, and the position sensing detector; wherein, a third frequency generator is positioned in connection to the first driver; a first frequency difference filter is positioned in connection to the first driver such that a first frequency difference is the difference of frequencies generated by the first frequency generator and the third frequency generator; a fourth frequency generator is positioned in connection to the second driver; a second frequency difference filter is positioned in connection to the second driver such that a second frequency difference is the difference of frequencies generated by the second frequency generator and the fourth frequency generator; the first frequency difference and the second frequency difference are established to be equal to each other, and the coherent detector is configured the first frequency difference and the second frequency difference at its output; an electrically controlled conjugating telescope is placed between the first beam splitter and the human eye in a refraction adjustable position to optically conjugate a plane of the retina with a plane of the position sensing detector and a plane of the coherent detector; a coherent detector is placed on a path of laser radiation after the first beam splitter with an aperture thereon in front of the coherent detector, the coherent detector with a low-pass filtering at its output; a phase discriminator is placed at the output of the coherent detector, the phase discriminator having two switchable inputs that are a first reference input connected to an output of the first frequency difference filter and a second reference input connected to the output of the second frequency difference filter; and the third frequency generator, the fourth frequency generator, the electrically controlled conjugating telescope, and the phase discriminator in operable communication with the processing unit.

In this embodiment, the first beam splitter may be a polarizing beam splitter configured to pass therethrough a linearly polarized light from the laser to the human eye and to reflect an orthogonal component of depolarized radiation from the human eye directing it to the position sensing detector and to the coherent detector. Further in this embodiment, the position sensing detector has a two-channel configuration corresponding to the first orthogonal direction and the second orthogonal direction of the position measurement.

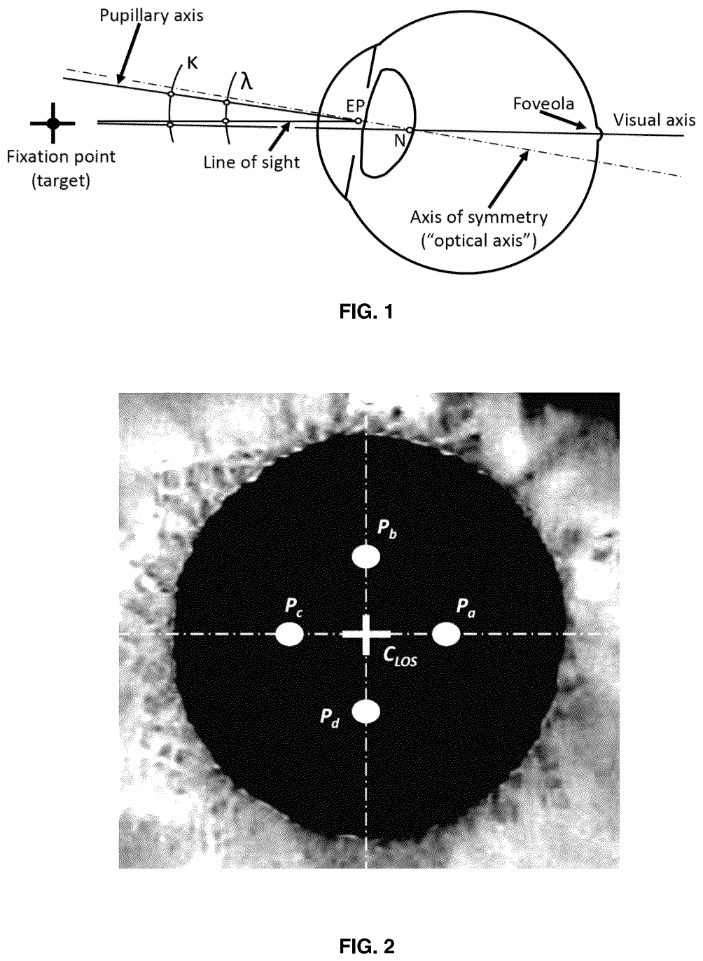

As mentioned above, the optical parameters of the eye could be described most adequately with reference to its visual axis that is defined as the line between a fixation point positioned in the infinity and the center of the foveola (FIG. 1). Intermediary points crossed by the visual axis are the first and the second nodal points, often treated as a single one (N) due to small (about a quarter of millimeter) distance between them, simply called a nodal point. The visual axis has also another name—the nodal axis. Because the nodal point can be defined only virtually, without anchoring to any visually perceptible sign or configuration, the visual axis is often replaced by the line of sight (LOS). The line of sight is defined as a line, connecting the central point of far vision (fixation point, target) with the center of the pupil. It is suggested for the majority of wavefront instruments that the center of the pupil coincides with the corneal vertex, designated as the center of a set of the first Purkinje images (the reflexes of four or six point sources of light, usually from LEDs—light emitting diodes).

As an example, FIG. 2 shows an eye pupil with a cross-marked center C_{LOS} of it, the cross mark being a geometrical center of an image of the Purkinje reflexes Pₐ, P_{b}, P_{c}, P_{d} of the lights from four infrared light emitting diodes. This point is suggested to be the point where the line of sight crosses the cornea.

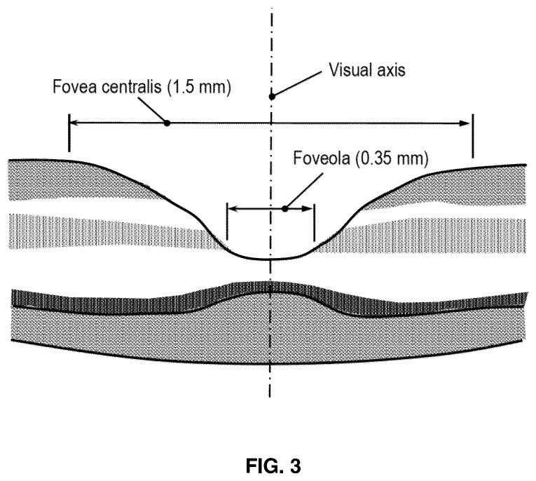

The visual axis crossing the center F of the foveola is shown in FIG. 3. The size of the pit is about 0.35 mm. The task of the first step of measurements is to find the direction on the point F in the position of the eye gazing at a point target in the infinity.

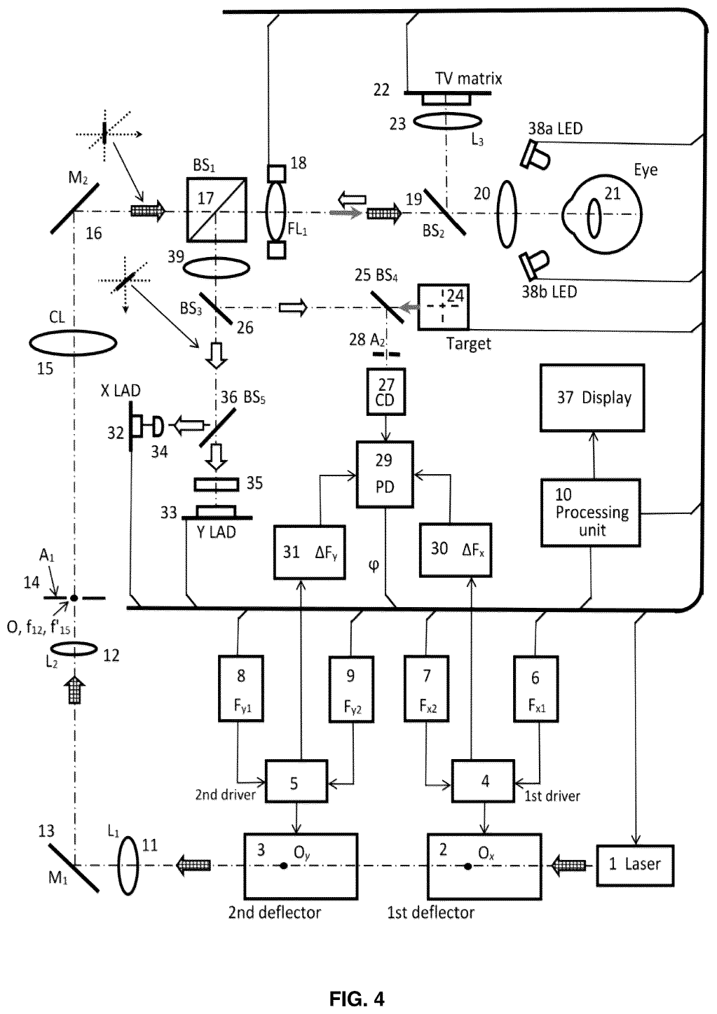

The means and procedures of the solution are described with the reference to the functional schematic of the device of FIG. 4. In this device, laser 1 is attached by its output to the input of the first deflector 2, having its center of scanning in point Oₓ. The second deflector 3 is mounted in series with the first deflector 2. The second deflector 3 has its center of scanning O_{y}. The first deflector 2 and the second deflector 3 can be of any type, but from the consideration of the speed of deflection and scanning, acousto-optical deflectors can be recommended based on the principles of light diffraction on the grating, created in acousto-optical crystals by the ultrasound waves.

For the purposes of the present invention, the centers of scanning in the orthogonal (X and Y) directions should coincide. This can be provided by a telescope translating the Oₓ center of scanning into the O_{y} center of scanning. Practically, this requirement may be neglected, and the deflectors 2 and 3 can be designed in the way to provide their crystals as near to each other as possible, in the order of millimeters. To create diffraction gratings in the crystals, drivers are used where the first driver 4 connected to the first deflector 2 is for deflecting the laser beam in X direction, and the second driver 5 connected to the second deflector 3 is for deflecting the laser beam in Y direction.

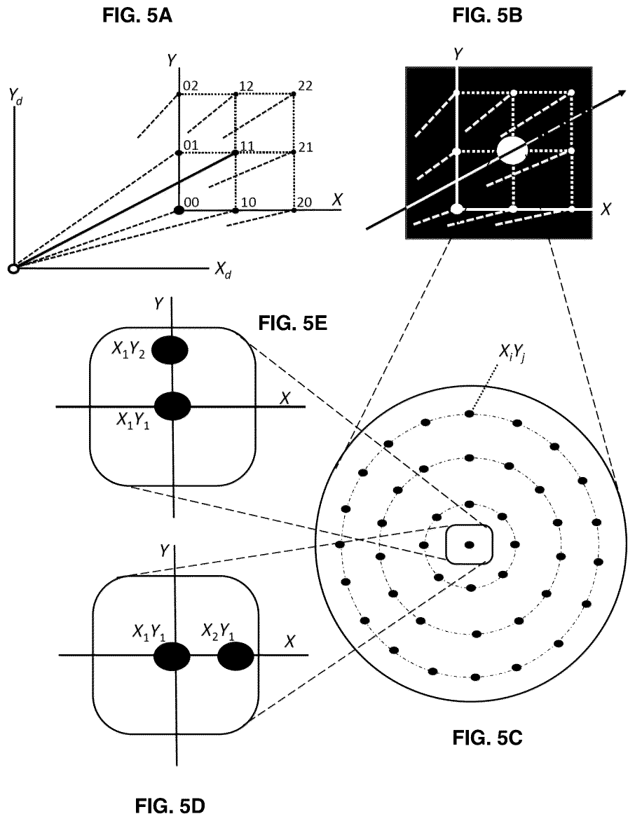

The angle of deflection depends on the grating spacing, defined by the frequency of the high-frequency signal applied to the transducer exciting the elastic wave in the crystal. FIG. 5A demonstrates the zeroth, the first and the second orders of diffraction. The zeroth order of diffraction in X direction crosses the nodes 00, 01, 02. The first order of diffraction in X direction crosses the nodes 10, 11, 12. The second order of diffraction in X direction crosses the nodes 20, 21, 22. Similarly, Y-th zero orders are 00, 10, 20. Y-th first orders are 01, 11, 21. Y-th second orders are 02, 12, 22. Only the first order of diffraction for both, X and Y directions crossing the node 11 is used in our device for angular orientation of the laser beam in these, X and Y directions.

The selection is made by any way of suppression of all orders, except the first one, for example, by a hole (aperture) in a non-transparent material, as shown in FIG. 5B. This process is called also the spatial filtration, and the mentioned aperture is called a spatial filter. The selected first-order beam can be scanned within the selecting aperture to create a sequence of directions for probing the eye (FIG. 5C) in the process of laser ray tracing used for reconstruction of the wavefront, or refraction distribution. For the purposes of this invention, a single laser beam can be split in X direction (FIG. 5D), or in Y direction (FIG. 5E), thus forming an X-split double beam, or a Y-split double beam. More detailed description of the implementation of these configurations are described below.

Each of the drivers 4 and 5 has two inputs (FIG. 4). The first input of the first driver 4 is connected to the output of the first generator 6 generating the frequency Fₓ₁. The first input of the second driver 5 is connected to the output of the second generator 8 generating the frequency F_{y1}. The second input of the first driver 4 is connected to the output of the third generator 7 generating the frequency Fₓ₂. The second input of the second driver 5 is connected to the output of the fourth generator 9 generating the frequency F_{y2}.

The drivers 4 and 5 can function in two modes: the mode of laser ray tracing and the mode of visual axis designation. In the mode of ray tracing, only the first input of the first driver 4 is connected to the output of the first generator 6. Similarly, only the first input of the second driver is connected to the output of the second generator 8. In the mode of visual axis designation, both inputs of the first driver 4 are connected to the outputs of both of the generators 6 and 7, as well as both inputs of the second driver 5 are connected to the outputs of both of the generators 8 and 9. The first driver 4 delivers two frequencies Fₓ₁ and Fₓ₂ to the first deflector 2 in the linear mode, i.e., without frequency conversion. Similarly, the second driver 5 delivers two frequencies F_{y1} and F_{y2} to the second deflector 3, also in the linear mode. Laser 1 and generators 6, 7, 8, and 9 are connected to a processing unit 10 to exchange with the information and with the control signals.

In other words, in the laser ray tracing mode, the device uses single beam scanning (FIG. 5C). In the mode of visual axis designation, the device uses double beam scanning (FIGS. 5D-5E), where the expression “double beam” means a beam derived from a single beam by the way of its splitting by the diffraction grating of the acousto-optical crystal. The beam splitting results also in the difference of carrier frequencies between the split beams.

The output of the second deflector 3 is directed into the telescope created by two lenses 11 (L₁) and 12 (L₂). The path of the laser beams into the eye is connoted by the texture-filled arrows. To make the design more compact, the optical path between the lenses 11 and 12 is bent by the mirror 13 (M₁). The centers of scanning Oₓ and O_{y} of each of the deflectors 2 and 3 are transferred into the space after the lens 12. In the assumption that Oₓ and O_{y} are managed to coincide or to be at so small distance between them, that it can be neglected, the center of scanning after the lens 12 can be designated as O, coinciding with the back focus f₁₂ of the lens 12. In the space of diffracted beams, its position must coincide with the direction of the first order of diffraction, both in X and in Y directions. Spatial filtering, selecting the first order of diffraction, is provided by the aperture 14 (A₁), being a hole, which size should be smaller than the distance between the orders of diffraction, practically, its diameter is of the order of a millimeter. The center of this aperture coincides with the point O.

The next optical element on the path of the laser beam is the collimating lens 15 (CL). Its front focus f′₁₅ coincides with the back focus f₁₂ of the lens 12, and as such, with the conjugated center of scanning O in the center of the aperture 14 (A₁). This design provides the direction parallel to the optical axis of the device for any beam after the collimating lens 15.

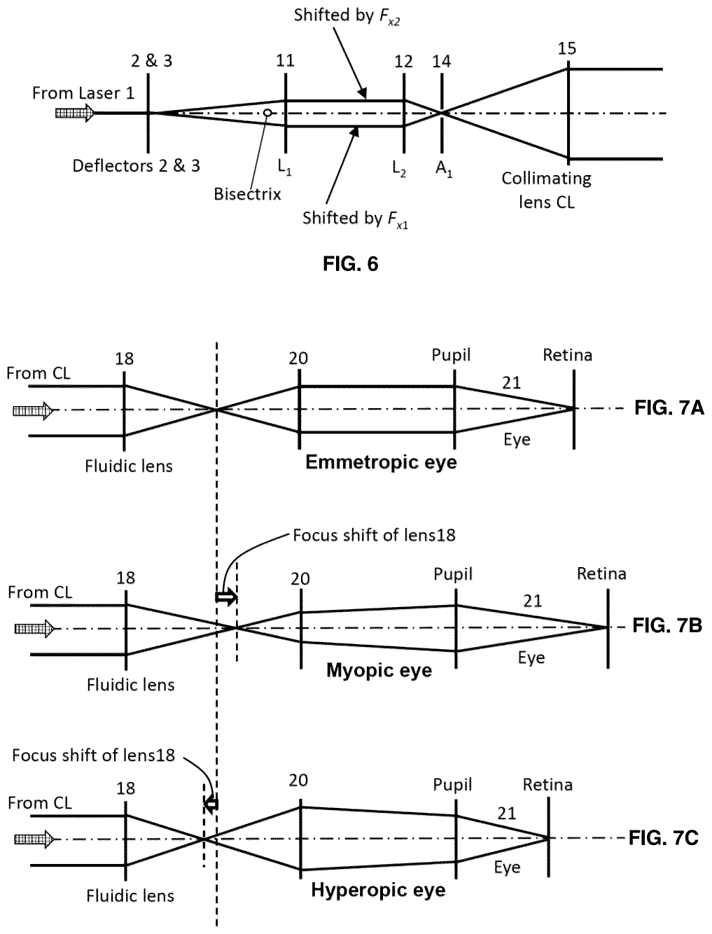

The evolutions of the laser beam along its path from laser 1 to the exit from the collimating lens 15 in the plane XOZ are shown in the schematic of FIG. 6. The same evolutions are made by the laser beam when it is split in Y direction, i. e., in the plane YOZ. The lenses in FIG. 6 are replaced by the principal planes of their thin equivalents. The bisectrix of the split beams exiting from the equivalent point of scanning Oₓ is extended along its path up to the final point on the retina. The same is valid when following from the point of scanning O_{y}.

For better clarity, no scaling is taken into account in the schematic. One of the beams is suggested to be split by the signal having the frequency Fₓ₁, the other one—by the signal having the frequency Fₓ₂ (similarly, F_{y1} and F_{y2} for the YOZ plane). Aperture 14 (A₁) playing the role of a spatial filter—selector of the first order of diffraction (order 11 in FIG. 5A) is positioned in the coinciding foci of lenses 12 and 15, thus creating parallel beams at the exit of the lens 15. It should be noted here that the similar evolutions/transforms are undergone by the beam when scaled for the wavefront measurement.

Coming back to FIG. 4, one can see that the mirror 16 (M₂) bends the beam direction towards the beam splitter 17 (BS₁), after which a switchable fluidic lens 18 is installed in sequence with a beam splitter 19 (BS₂) and a lens 20 on the path to the eye 21.

The bean splitter 17 can be of any kind, but for the purpose of this invention, an optimal or representative example would be a polarizing beam splitter. It allows the vertical linear polarization of the laser beam from the last stage of deflectors to continue its path in the direction of the eye. The same vertical linear polarization has the laser 1. It is a feature of a cell of the acousto-optical deflector to rotate the linear polarization 90 degrees. It means that two sequential cells return back the initial type of polarization.

The paths of laser beams after the collimating lens 15 (CL) to the eye 21 are shown in FIG. 7. Three kinds of path features are demonstrated: FIG. 7A—for an emmetropic eye (fluidic lens is in its initial position corresponding to the requirement of afocality of the telescope consisting of lenses 18 and 20 (afocality condition: the back focus of lens 18 and the front focus of lens 20 must coincide); FIG. 7B—for a myopic eye (focus of the lens 18 is shifted to a longer value); FIG. 7C—for a hyperopic eye (focus of the lens 18 is shifted to a shorter value); in both (FIGS. 7B-7C) cases ametropia of the eye is to be compensated.

To achieve the vergence conjugation consisting in the compensation of ametropia, the optical powers (one of them—of the fluidic lens 18 is variable), the distance between the lenses 18 and 20, and the distance from the lens 20 to the eye 21 are to be optimized by the mathematical procedures described in the publication of V. Molebny et al. “Defocus compensation with fluidic optics in a raytracing wave front sensor”, Proc. 7 European/1 World Meeting on Visual and Physiological Optics. Wroclaw, 2014, pp. 222-225. This conjugation should be made both for measurement of the wavefront and for visual axis designation.

The path back (FIG. 4) from the eye 21 to a TV matrix 22 goes through the lens 20, the beam splitter 19, and an objective lens 23 (L₃). The sensitive surface of the TV matrix 22 is conjugated with the pupil of the eye with the lenses 20 and 23 which form an objective lens for the matrix.

Preliminary alignment of the patient's eye 21 and its positioning is provided using a target 24. The path of its image to the eye (filled arrows) is through beam splitters 25 (BS₃), 26 (BS₄), lens 39, beam splitter 17 (BS₁), fluidic lens 18, beam splitter 19 (BS₂), and the lens 20.

For determination of the direction on the deepest point of the foveola, a coherent detector 27 is installed on the path of the light (empty arrows) from the eye 21 going through the lens 20, beam splitter 19, fluidic lens 18, beam splitter 17, lens 39, beam splitters 26 and 25, and the aperture 28 (A₂). The output of the coherent detector 27 includes a low-pass filter at its output and is connected to the signal input of a phase discriminator 29 (PD). The role of the beam splitter 17 consists in reflecting the orthogonal polarization to the detection community.

The phase discriminator 29 has also two other inputs: the first reference input and the second reference input. The first reference input is connected to the output of the first frequency difference unit 30, and the second reference input is connected to the output of the second frequency difference unit 31. The input of the unit 30 is connected to the first driver 4, and the input of the unit 31 is connected to the second driver 5. The output of the phase discriminator 29 is connected to the processing unit 10. The phase discriminator 29 has the feature of measuring the phase shift of the signal from the coherent detector 27 relatively the reference signal ΔFₓ when measuring the height difference of pit slopes along X direction, and relatively the reference signal ΔF_{y} when measuring the height difference of pit slopes along Y direction. Switching between these two data is controlled by the processing unit 10.

The reference signals delivered to the phase discriminator 29 are the signals with the frequency difference of Fₓ₁ and Fₓ₂ equal to ΔFₓ (when defining the position along X coordinate), or the frequency difference of F_{y1} and F_{y2} equal to ΔF_{y} (when defining the position along Y coordinate). The frequency difference ΔFₓ is derived from the frequency difference unit 30, and the frequency difference ΔF_{y} is derived from the frequency difference unit 31. In general, ΔFₓ may be different than ΔF_{y}, but for the sake of simplification of the processing, they are recommended to be equal: ΔFₓ=ΔF_{y}=ΔF.

To measure the coordinates of an image of the laser spot on the retina, a position sensing unit of detectors is used. Several versions of such units are well known. One of the options is to measure X and Y coordinates by separate X and Y sensors based on linear array detectors 32 and 33, any of them having an array of photosensitive diodes positioned in line oriented along the X axis to measure the X coordinate, and along the Y axis when measuring the Y coordinate. Stretching the image of the laser spot by the cylinder lenses 34 and 35, in case it is defocused, is made in the direction, orthogonal to the orientation of the diode rows. The linear arrays 32 and 33 are connected by their outputs to the processing unit 10. The path of the light scattered in the eye 21 and coming from it is connoted by empty arrows. To reach the linear array 33 it passes the lens 20, beam splitter 19, fluidic lens 18, beam splitter 17, lens 39, beam splitter 26, beam splitter 36, cylinder lens 35. Up to the beam splitter 36, the path to the linear array 32 is the same, but on the beam splitter 36, the path is turned to the cylinder lens 34.

In front of the eye, a set of light emitting diodes (LEDs) 38a, 38b, etc., is positioned providing the Purkinje reflexes (FIG. 2) from the reflecting surface of the eye, of which, the reflexes from the first surface of the cornea are the most significant for functioning of the proposed device.

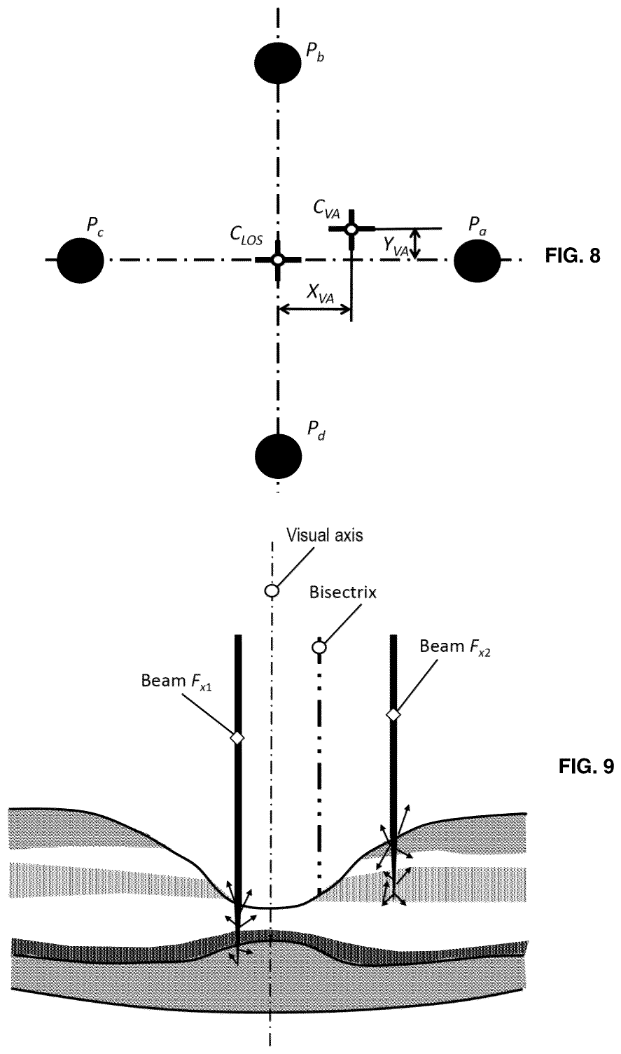

The implementation of the proposed method and device comprises the following operations. At the beginning, with the patient's eye gazing at the far target, the optical axis of the instrument should be oriented to the pupil's vertex whose position in the image of the eye is the cross-marked center C_{LOS} of a set of Purkinje reflexes Pₐ, P_{b}, P_{c}, P_{d}, crossed by the line of sight (FIG. 2). The next step should be the designation of the direction to the center of the foveola and defining the coordinates (X_{VA}, Y_{VA}), dislocated in the plane of the pupil relatively the point Cₗₒₛ(0,0), to present the point C_{VA}(X_{VA}, Y_{VA}), where the visual axis crosses the cornea (FIG. 8). These coordinates can be calculated in any other system of coordinates anchoring this point to the visually perceptible features of the pupil. All the rest of the ray tracing procedure is to be exercised with the optical axis of the instrument oriented along the designated visual axis, i.e., directed to cross the cornea in the dislocation-defined point C_{VA}(X_{VA}, Y_{VA}).

The operations with the device will follow in two steps: step 1—designation of the visual axis, step 2—measuring the wave front.

Step 1. Designation of the visual axis:

The image of the eye created by the TV matrix 22 and displayed on the display 37 is used to position the patient's eye so that its pupil is centered relatively the optical axis of the device. It is supposed that the center of the pupil coincides with its vertex which is designated by the center of the figure consisting of the images of Purkinje reflexes. The patient is asked to gaze his/her sight at the image of the target 24. To get the image of the target 24 in focus, the conjugation of the planes of the retina and the target is made by changing the optical power of the fluidic lens 18. Lenses 18 and 20 are designed to form a bi-telecentric system, i.e., telecentric in both image and object spaces within the range of the to-be-measured eye ametropia, usually from minus 10 to plus 10 diopters. If this condition is not met, scaling coefficients should be taken into account when calculating the eye's refraction.

Two options are possible to adjust the fluidic lens 18 to compensate for the eye's ametropia: either using the patient's commands by a joystick, for example, or by measuring the defocus component of Zernike series having made a preliminary single-beam ray tracing, i. e. with switched on generators 6 (frequency Fₓ₁) and 8 (frequency F_{y1}) and switched off generators 7 (no frequency Fₓ₂) and 9 (no frequency F_{y2}).

After the fluidic lens 18 has compensated the eye's defocus, double beam procedure starts to define the position of the eye to be oriented along its visual axis. Defining the direction toward the deepest point of the foveola pit is made separately for X and Y coordinates. For the beam doublet split in X direction, the procedure is as follows. The second driver 5 functions in the single beam mode (only the signal of frequency F_{y1} is applied to the crystal), and the first driver 4 functions in the double-beam mode (the signals of two frequencies Fₓ₁ and Fₓ₂ are applied to the crystal. With this combination of driver functioning, a double beam split in X direction can be positioned at any point in the zone of the foveola (FIG. 9) by controlling the frequencies Fₓ₁ (to the left in the drawing) and Fₓ₂ (to the right in the drawing). The bisectrix is shown here as the axis equally outlying from both laser beams. The splitting angle corresponds to the frequency difference ΔFₓ. Similarly, with the first driver 4 in the single beam mode (only the signal of frequency Fₓ₁ is applied to the crystal) and the second driver 5 in the double-beam mode (the signals of two frequencies F_{y1} and F_{y2} are applied to the crystal), a double beam split in Y direction can be positioned at any point in the zone of the foveola by controlling the frequencies F_{y1} and F_{y2}. Angular difference corresponds to the frequency difference ΔF_{y}. The process of scatter in the tissues of eye's bottom is illustrated in FIG. 9. A part of laser light penetrates into the depth of the tissue where it is scattered including the back direction. The back scattered radiation propagates to be detected by the coherent detector 27.

The search in the X direction for the deepest point in the foveola pit starts with a doublet of beams directed along the patient's line of sight to the foveola. Backscattered light is received by the coherent detector 27 through the aperture 28 whose size is a compromise between the amount of light (the more, the better) and the number of interfering fringes (the less, the better). Practically it has a diameter of the millimeter range. Coherent detector 27 implies a low-pass filter at its output, resulting in the difference frequency ΔFₓ at the detector's output. The phase difference φₓ at this frequency carries the information about the path difference between the beams in X direction. The signal from the output of the coherent detector is delivered to the signal input of the phase discriminator 29, to the reference input of which, an X reference signal of the frequency ΔFₓ is delivered from the frequency difference unit 30. The signal proportional to the phase difference ox is directed to the processing unit 10 that calculates what next position of the beam doublets should be to get the phase difference corresponding to the equal paths of both beams, that means that their bisectrix is pointing toward the middle of the foveola pit. The procedure of beam repositioning takes several tens of microseconds, so the adjustment of the doublet orientation takes less that a millisecond.

The phase difference O_{y} at the frequency ΔF_{y} carries the information about the path difference between the beams in Y direction. The signal from the output of the coherent detector is delivered to the signal input of the phase discriminator 29, to the reference input of which an Y reference signal of the frequency ΔF_{y} is delivered from the frequency difference unit 30. The signal proportional to the phase difference φ_{y} is directed to the processing unit 10 that calculates what next position of the beam doublets should be to get the phase difference corresponding to the equal paths of both beams, that means (similarly to the X direction) that their bisectrix is pointing toward the middle of the foveola pit in Y direction. In total, the procedure of beam repositioning for both directions is limited by several milliseconds.

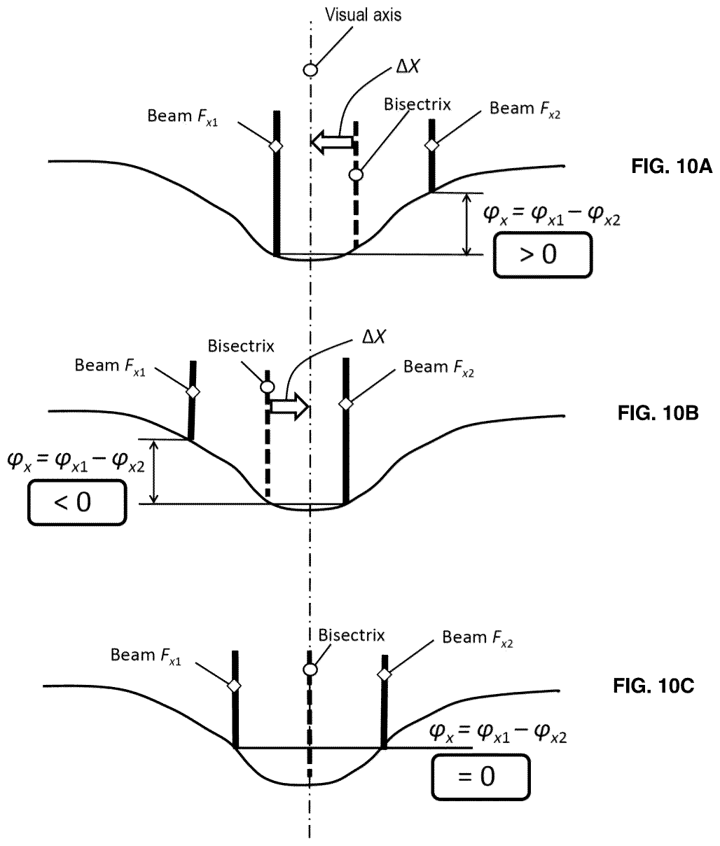

The process of the search of the visual axis is illustrated by FIGS. 10A-10C. Three cases are shown for the plane XOZ:

FIG. 10A—the beam diffracted on the grating created by using the frequency Fₓ₁ has longer path than that diffracted on the grating created by using the frequency Fₓ₂, therefore its phase delay φₓ₁ will be longer than the delay φₓ₂. The phase difference φₓ=φₓ₁−φₓ₂ converted into the phase difference at the frequency ΔFₓ will be positive (φₓ22 0) meaning that the error signal controlling the frequencies Fₓ₁ and Fₓ₂ should make both beams to move synchronously to the left bringing the bisectrix nearer to the visual axis. To do so, the measured phase difference φₓ is recalculated by the processing unit 10 into the to-be-corrected frequency values Fₓ₁, Fₓ₂ with keeping ΔFₓ constant.

FIG. 10B-the beam diffracted on the grating created by frequency Fₓ₂ has longer path than that diffracted on the grating created by frequency Fₓ₁, therefore its phase delay φₓ₂ will be longer than the delay φₓ₁. The phase difference φₓ=φₓ₁−φₓ₂ converted into the phase difference at the frequency ΔFₓ will be negative (φₓ<0) meaning that the error signal controlling the frequencies Fₓ₁ and Fₓ₂ should make both beams to move synchronously to the right bringing the bisectrix nearer to the visual axis.

FIG. 10C—the beam diffracted on the grating created by both frequencies Fₓ₁ and Fₓ₂, have equal paths to the opposite slopes of the pit, therefore their phase delays φₓ₁ and φₓ₂ will be equal to each other. The phase difference φₓ=φₓ₁−φₓ₂ converted into the phase difference at the frequency ΔFₓ will be equal to zero (φₓ=0) meaning that the bisectrix coincides with the visual axis. A similar search should be executed for the plane YOZ. In this plane, the frequencies F_{y1}, and F_{y2} are to be varied keeping ΔF_{y} constant.

As mentioned above, it is expedient to have the same value for the frequency difference ΔFₓ for X channel and ΔF_{y} for Y channel: ΔFₓ=ΔF_{y}=ΔF. This makes it possible to have the low-pass filter the same for both channels. Otherwise, two different filters should be switchable at the output of the coherent detector 27. This peculiarity does not influence the principles of this invention.

### Step 2. Measuring the Wavefront:

With the established dislocation position X_{VA}, Y_{VA} of the cornea crossing point by the visual axis, the final measurement of the refraction is made. The laser beam is directed to the eye through the first and through the second deflectors 2 and 3, driven by the first and the second drivers 4 and 5, both functioning in the single-beam mode, i. e., the first driver 4 being controlled only by the first generator 6 (frequency Fₓ₁), and the second driver 5 being controlled only by the second generator 8 (frequency F_{y1}). Generators 7 (frequency Fₓ₂) and generator 9 (frequency F_{y2}) are silent during the final measurement of the refraction. Therefore, no beam splitting is performed. The beams are positioned in any time sequence and any space layout within the eye aperture, necessary to reconstruct the refractive parameters of the eye.

The beam follows to the eye along the path described above through the lenses 11, 12, spatial filter (aperture) 14, collimating lens 15, beam splitter 17, fluidic lens 18, beam splitter 19, and lens 20. Lenses 18 and 20 designed in a way described above. Into account is taken also the condition of conjugation of the plane of the retina with the target 24, sensitive surface of the coherent detector 27, and sensitive surfaces of linear array detectors 32 and 33. The cylinder lenses 34 and 35 serve for compressing the image of the laser spot on retina. In the conjugated position of the lenses 18-20, the image of the spot will be stretched a little bit in the perpendicular direction.

After the laser spot on retina is imaged on the linear array detectors 32 and 33, its coordinates are determined by the processing unit 10. From the known coordinates of crossing the pupil and hitting the retina with the knowledge of the parameters of the imaging optics, the refraction power of the eye for each point of the pupil is calculated by the processing unit 10. This information is enough to reconstruct the 2D distribution of these and derivative parameters of the eye and displaying them on the display 37.

What is claimed is:

1. A method for measuring a wave front in a human eye with respect to its objectively determined visual axis, comprising:
probing the eye with a laser beam consecutively in time in a set of points within a pupil of the eye;
detecting radiation backscattered from a retina in the eye;
measuring coordinates of laser spots on the retina; and
reconstructing the wave front from a refraction distribution over the pupil of the eye.

2. The method of claim 1, wherein prior to the probing step, the method comprises determining the visual axis in the eye.

3. The method of claim 2, wherein determining the visual axis comprises:
splitting the laser beam into a beam doublet separately in X and Y directions;
probing the retina in a zone of a foveola pit with the beam doublet in both the X and Y directions of beam splitting;
detecting the laser light backscattered from the zone of the foveola pit;
measuring and comparing a phase shift of the beams in the beam doublet relative to each other in both of the X and Y directions of beam splitting;
determining the direction of the beam doublet in a position of equal depths of opposite slopes of the foveola pit indicated by a zero value of the phase shift between the beams in the beam doublet; and
designating the visual axis as a bisectrix of said beam doublet in the position of equal depths of the opposite slopes of the foveola pit for both of the X and Y directions of beam splitting.

4. The method of claim 3, further comprising orienting the laser beam along the visual axis of the eye during measurement of the wave front.

5. The method of claim 3, wherein the beam splitting comprises diffracting in a two-axis acousto-optical deflector separately in orthogonal directions in a plane of the pupil.

6. The method of claim 3, wherein the opposite slopes of the foveola pit have a depth difference thereon defined as a phase difference between carrier frequencies of said split beams converted to a phase difference between signals at a frequency difference between the carrier frequencies of said beam doublet.

7. The method of claim 3, wherein the probing with said beam doublet is varied directionally until a value of the phase difference becomes zero for both orthogonal directions.

8. A device for a wave front measurement in a human eye with respect to its objectively determined visual axis, comprising:
a laser configured to emit a laser beam of a wavelength suitable for ray tracing;
a pair of diffraction-based deflectors composed of acousto-optical crystals and comprising a first deflector oriented to deflect said laser beam in a first direction orthogonal to the laser beam propagation and a second deflector oriented to deflect said laser beam in a second direction orthogonal to the first direction, each of said first deflector and said second deflector in the pair having an entrance aperture and an exit aperture and each of said first deflector and said second deflector in the pair positioned along a path of the laser beam such that their effective centers of deflection substantially coincide;
a pair of drivers comprising a first driver operably connected to a first frequency generator and a second driver operably connected to a second frequency generator, each of said first driver and said second driver in the pair configured to acousto-optically drive the first deflector and the second deflector, respectively;
a telescope and a collimating lens placed sequentially in optical alignment with the centers of deflection of said first deflector and said second deflector in the pair of deflectors;
a first beam splitter placed in the path of the laser beam to the human eye and of the reflected radiation from the human eye;
a position sensitive detector placed in the path of the reflected radiation from the human eye; and
a processing unit in operable communication with at least the laser, the first frequency generator, the second frequency generator, and the position sensitive detector;
wherein,
a third frequency generator is positioned in connection to said first driver;
a first frequency difference filter is positioned in connection to said first driver;
a fourth frequency generator is positioned in connection to said second driver;
a second frequency difference filter is positioned in connection to said second driver;
an electrically controlled conjugating telescope is placed between the first beam splitter and the human eye in a refraction adjustable position to optically conjugate a plane of the retina with a plane of the position sensitive detector and a plane of the coherent detector;
a coherent detector is placed on a path of laser radiation after the first beam splitter with an aperture thereon in front of said coherent detector, said coherent detector with a low-pass filtering at its output;
a phase discriminator is placed at the output of the coherent detector, said phase discriminator having two switchable inputs that are a first reference input connected to an output of the first frequency difference filter and a second reference input connected to the output of the second frequency difference filter; and
said third frequency generator, said fourth frequency generator, said electrically controlled conjugating telescope, and said phase discriminator in operable communication with the processing unit.

9. The device of claim 8, wherein the first beam splitter is a polarizing beam splitter configured to pass therethrough a linearly polarized light from the laser to the human eye and to reflect an orthogonal component of depolarized radiation from the human eye directing it to the position sensitive detector and to the coherent detector.

10. The device of claim 8, wherein the position sensitive detector has a two-channel configuration corresponding to the first orthogonal direction and the second orthogonal direction of the position measurement.

11. The device of claim 8 wherein a first frequency difference is the difference of frequencies generated by the first frequency generator and the third frequency generator.

12. The device of claim 11, wherein a second frequency difference is the difference of frequencies generated by the second frequency generator and the fourth frequency generator.

13. The device of claim 12, wherein said first frequency difference and said second frequency difference are established to be equal to each other, and the coherent detector is configured to pass the first frequency difference and the second frequency difference at its output.
